(19) United States
(12) Patent Application Publication
(10) Pub. No.: US 2021/0252339 A1
(43) Pub. Date: Aug. 19, 2021

(54) AUGMENTED REALITY FOR DETECTING ATHLETIC FATIGUE

(71) Applicant: Strive Tech Inc., Bothell, WA (US)

(72) Inventors: Nikola Mrvaljevic, Bothell, WA (US); Carsten Gabriel Winsnes, Bothell, WA (US)

(73) Assignee: Strive Tech Inc., Bothell, WA (US)

(21) Appl. No.: 17/270,977

(22) PCT Filed: Aug. 21, 2019
(86) PCT No.: PCT/US19/47487

§ 371 Date: Feb. 24, 2021

Related U.S. Application Data
(60) Provisional application No. 62/722,763, filed on Aug. 24, 2018.

(51) Int. Cl. A63B 24/00; A63B 71/06; G06N 20/00; G16H 40/63

(52) U.S. Cl. CPC A63B 24/0062; A63B 71/0622; G06N 20/00; G16H 40/63; A63B 2220/807; A63B 2220/05; A63B 2024/0068; A63B 2225/50; A63B 2230/06; A63B 2220/40; A63B 2230/60; A63B 2071/0625; A63B 2220/836

(57) ABSTRACT

An augmented reality system and method of using the same for real-time assessment of athletic performance is described. The system includes a digital platform, itself including a display, at least one camera, and a communications module. The system further includes a performance monitor carried by a garment and configured to communicate wirelessly with the digital platform, a logic engine, and an interactive user interface, presenting real-time data and images of athletic performance. The real-time data and images include images obtained by the at least one camera and athletic performance data collected via the wearable sensor system. The augmented reality system provides a real-time augmented reality environment combining analysis of performance with live images of a subject of observation.

### BACKGROUND

Market research and subject matter experts are showing that fatigue can make an athlete more susceptible to injury, and may, in fact, be one of the leading causes of injuries. Thus, there is a need to detect the onset of fatigue while an athlete is actively training, conducting practice, or participating in a live game. When a trainer (e.g., an athletic trainer or coach) detects signs of fatigue, the trainer can intervene to reduce the likelihood of fatigue-related injury. For example, when a trainer detects fatigue, the trainer may instruct the athlete to slow down or focus on technique. In addition or alternately, a trainer may pull the athlete from a game or a practice session for rest and recovery.

One of the challenges with detecting fatigue is that traditional methods of monitoring athletic performance, such as real-time heart rate monitoring, do not in and of themselves necessarily indicate when an athlete is fatigued. For example, an athlete experiencing lack of sleep, may still exhibit a normal or expected heart rate during a practice session, but may experience earlier on-set of fatigue due to lack of sleep. As a result, the athlete may have a heightened susceptibility to fatigue-induced injury that the trainer may be unaware of because the athlete's heart rate appeared to be normal.

Precise control of stance, posture, and movement improves the effectiveness of exercise routines and prevents injury. Typically, an expert, either a coach, a trainer, or a doctor, will directly observe a subject, such as an athlete or a patient during the exercise, and will make real time corrections based on a number of intuitive factors. This approach is limited in that the expert can only work in real-time while the subject is under observation. In most team and clinic environments, an expert must observe a number of subjects simultaneously, making fine adjustments to stance and posture based only on information collected during the short period of time that each subject is under observation.

Accordingly, there remains a need for efficient and reliable injury prediction systems and methods that aim to address one or more problems of prior art systems.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Analytics systems configured in accordance with various embodiments of the present technology, can address at least some limitations of traditional methods of detecting fatigue and/or monitoring athletic performance. As described below, the system can provide analytics in an augmented reality environment that are real-time, comparative, and predictive in nature, and can detect the early onset of fatigue which may not be readily detectable by visual monitoring alone. This, in turn, provides the opportunity for improved training outcomes, and earlier intervention and corrective action to reduce the risk of fatigue-related injuries.

Various embodiments of the present technology include an augmented reality system (such as, a real time analytics system) incorporating data collected from wearable sensor technology, also referred to as a performance monitor, into an interactive user interface having a receiver (such as, a wireless receiver) for data. In some embodiments, the interactive user interface provides an augmented reality display of health- and/or performance-analytics data integrated into a video image of a subject. The interactive user interface may present a subject under observation during an athletic performance as viewed through a camera in a mobile device. The user interface may present data collected over a period of time preceding the athletic performance, thereby providing in-depth information on athletic development, therapeutic efficacy of an exercise routine, recovery after a sports injury, etc. In different embodiments, the inventive technology may be used for other purposes. For example, the inventive technology may be used for military training or in conjunction with consumer devices.

In some embodiments, the user interface may communicate with a data storage system including a processor implementing machine learning analytics. The interactive user interface may be implemented on a digital platform that analyzes real-time data collected from the wearable sensor technology as the subject exercises or rests, and may compare the collected data with aggregated data collected from additional subjects and subsequently analyzed by a machine learning system. The machine learning analytics may implement predictive models to adjust the augmented reality display, indicating training information, such as likelihood of injury, asymmetric exertion, motion or posture irregularities, etc.

As understood by one of ordinary skill in the art, a “data storage system” as described herein may be a device configured to store data for access by a computing device. An example of a data storage system is a high-speed relational database management system (DBMS) executing on one or more computing devices and being accessible over a high-speed network. However, other suitable storage techniques and/or devices capable of quickly and reliably providing the stored data in response to queries may be used, and the computing device may be accessible locally instead of over a network, or may be provided as a cloud-based service. The data storage system may also include data stored in an organized manner on a computer-readable storage medium.

In some embodiments, the user interface is implemented in a mobile device. As such, the mobile device may be repositioned to observe multiple subjects during a given period of time. The user interface may facilitate various techniques to identify the subject presently under observation. For example, the user interface may implement facial recognition routines to identify a subject. Alternatively or additionally, the user interface may communicate via radio-frequency identification and/or Bluetooth with the performance monitor worn by the subject. For example, the performance monitor may include a radio frequency identifier (RFID) or other unique identifier that allows the analytics system to attribute newly collected data and to request previously collected performance data.

In some embodiments, the inventive technology includes an augmented reality system for real-time assessment of an athletic performance. In some embodiments, the system includes a digital platform. In an aspect, the digital platform includes a display, at least one camera, and a communications module. In some embodiments, the system includes a performance monitor carried by a garment and configured to communicate wirelessly with the digital platform, a logic engine, and an interactive user interface. In an aspect, the interactive user interface presents real-time data and images of the athletic performance in an augmented reality environment. In an aspect the real-time data and images include images obtained by the at least one camera and athletic performance data received from the performance monitor.

In an aspect, the interactive user interface further presents historical performance data and aggregated performance data.

In an aspect, historical performance data includes real-time data collected from an identified individual over a period of time.

In an aspect, aggregated performance data includes real-time data collected from a plurality of anonymized individuals.

In an aspect, the augmented reality system includes at least one predictive model indicating a likelihood of one or more of fatigue or injury in correlation to real-time data.

In an aspect, the performance monitor includes a plurality of sensors positioned in one or more regions of the garment configured to measure signals generated during the athletic performance and a performance monitor controller. In an aspect, the performance monitor controller includes an onboard analytics module configured to receive and process signals from the plurality of sensors and an onboard communications module in wireless communication with the digital platform.

In an aspect, the performance monitor includes sensors to measure orientation, acceleration, heart response, and muscle response.

In an aspect, the logic engine includes an implementation of machine learning.

In an aspect, the augmented reality system further includes a data storage system in communication with the digital platform and the performance monitor, the data storage system including an implementation of machine learning.

In some embodiments, the inventive technology includes a method of assessing athletic performance in real-time through an augmented reality environment. In some embodiments, the method includes selecting a subject of observation, identifying the subject of observation using a digital platform, presenting an augmented reality environment including an interactive user interface and data. In an aspect, the interactive user interface and data include images of the subject of observation collected via a camera and real-time data collected via a performance monitor. In some embodiments, the method includes receiving commands from a user via the interactive user interface, wherein the commands modify one or more of the interactive user interface, the operation of the performance monitor, the selection of the subject of observation, and the presentation of data.

In an aspect, the data further includes historical performance data collected from the subject of observation and aggregated performance data collected from multiple anonymized subjects.

In an aspect, the method further includes accessing real-time analytics provided by an external data storage system and processing the real-time data using model-predictions of athletic performance.

In an aspect, the method further includes identifying multiple subjects engaging in simultaneous athletic performances, presenting one or more available subjects via the interactive user interface, and prompting a selection of one or more of the available subjects for observation in real-time.

In an aspect, the method further includes indicating, via a visual or auditory signal, when the subject of observation has a high likelihood of adverse outcome from athletic performance.

In some embodiments, the inventive technology includes an augmented reality system for real-time assessment of a physical rehabilitation treatment. The augmented reality system may include a digital platform including a display, at least one camera, and a communications module, a performance monitor carried by a garment and configured to communicate wirelessly with the digital platform, a logic engine, and an interactive user interface. The interactive user interface may present real-time data and images of the physical rehabilitation treatment in an augmented reality environment. In an aspect, the real-time data and images include images obtained by the at least one camera and physical rehabilitation treatment data received from the performance monitor.

In an aspect, the interactive user interface further presents historical performance data and aggregated performance data. In an aspect, historical performance data includes real-time data collected from an identified individual over a period of time. In an aspect, aggregated performance data includes real-time data collected from a plurality of anonymized individuals.

In an aspect, the augmented reality system further includes at least one predictive model indicating a likelihood of one or more of fatigue or injury in correlation to real-time data.

In an aspect, the performance monitor includes a plurality of sensors positioned in one or more regions of the garment configured to measure signals generated during the athletic performance and a performance monitor controller. The performance monitor controller may include an onboard analytics module configured to receive and process signals from the plurality of sensors and an onboard communications module in wireless communication with the digital platform.

In an aspect, the performance monitor includes sensors to measure orientation, acceleration, heart response, and muscle response.

In an aspect, the logic engine includes an implementation of machine learning.

In an aspect, the augmented reality system further includes a data storage system in communication with the digital platform and the performance monitor, the data storage system including an implementation of machine learning.

### DESCRIPTION OF THE DRAWINGS

The foregoing aspects and attendant advantages of the inventive technology will become more readily appreciated as the same become better understood by reference to the following detailed description, when taken in conjunction with the accompanying drawings, wherein:

FIG. 1A is an augmented reality system in accordance with the present disclosure.

FIG. 1B illustrates facial recognition in accordance with the present disclosure.

FIGS. 2A-B illustrates biometric data in accordance with the present disclosure.

FIG. 3A is a mobile device in accordance with the present disclosure.

FIGS. 3B-D is a performance monitoring system in accordance with the present disclosure.

FIGS. 4 A-D illustrate a system in accordance with the present disclosure.

FIG. 5 is a flowchart of a method of assessing athletic performance in accordance with the present disclosure.

### DETAILED DESCRIPTION

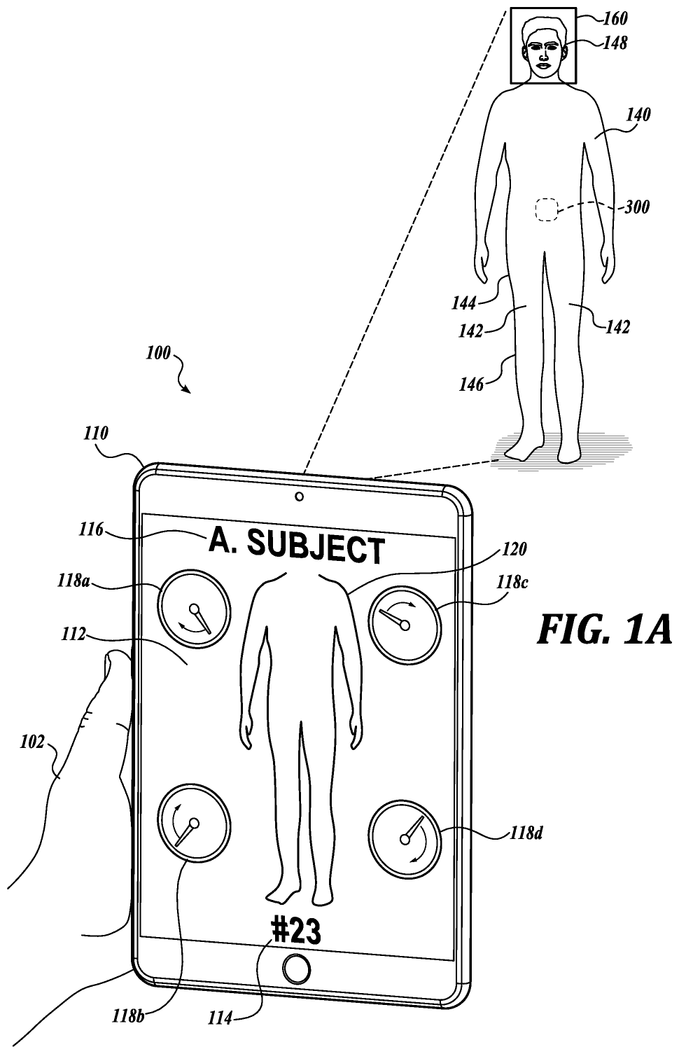

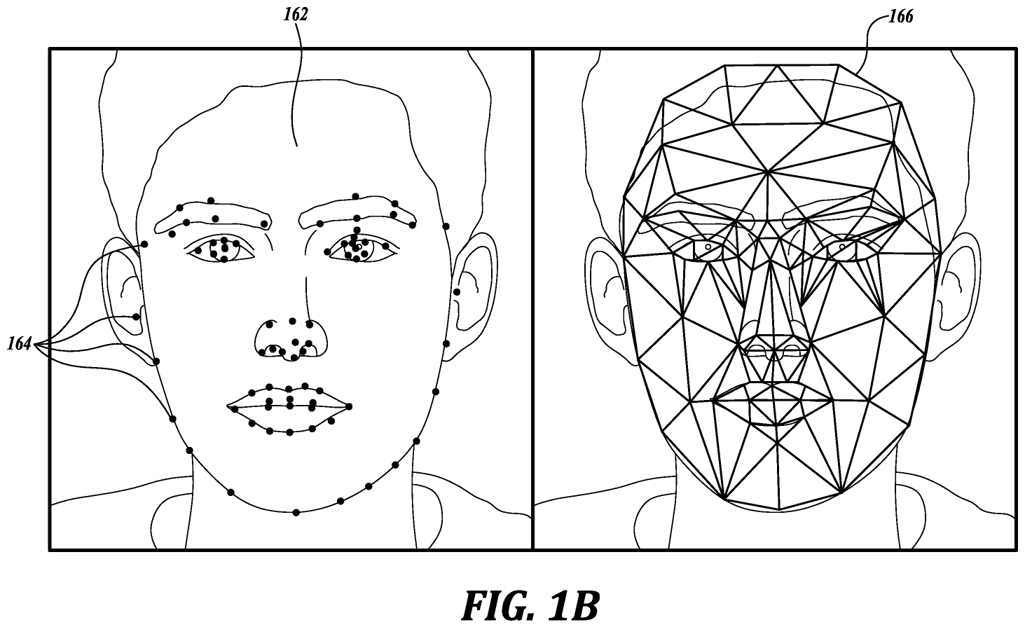

The following disclosure describes various embodiments of systems and associated methods for preparing personalized cosmetic formulas. A person skilled in the art will also understand that the technology may have additional embodiments, and that the technology may be practiced without several of the details of the embodiments described below with reference to FIG. 1-5.

FIG. 1A is a schematic diagram of an augmented reality system 100 in accordance with the present disclosure. In an embodiment, the augmented reality system 100 includes a digital platform 110, an interactive user interface 112, an anonymized identifier 114, a direct identifier 116, an augmented reality display including images 120 and multiple types of data, such as real-time data 118a, selected performance data 118b, historical performance data 118c, and aggregated performance data 118d (collectively referred to as “data” 118), and a subject 140 wearing a performance monitor 300, described in more detail below, including sensors measuring muscle groups of interest, such as a leg muscle 142, a glute muscle 144, and a hamstring 146.

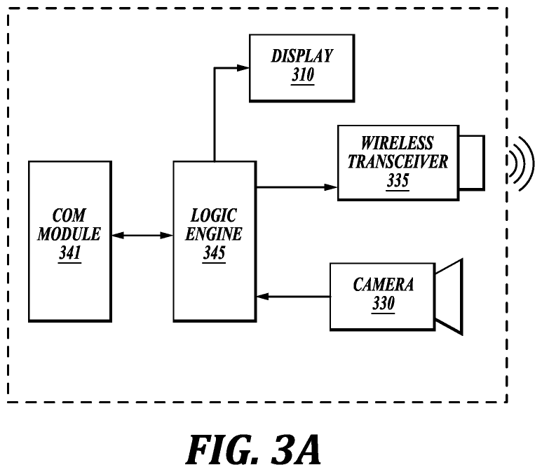

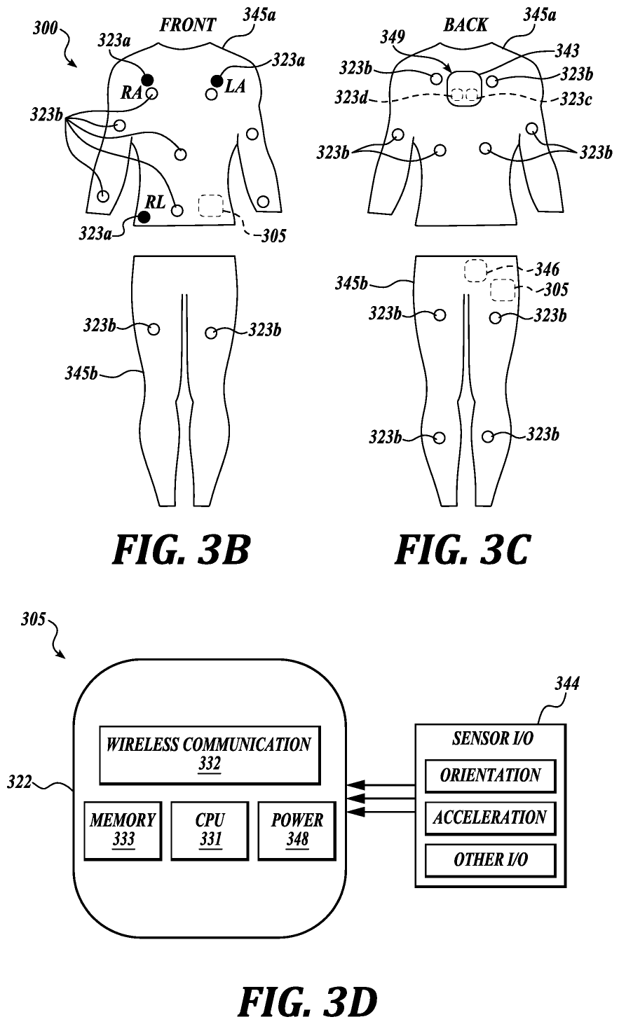

In some embodiments, the interactive user interface 112 includes an augmented reality display including real-time data 118a of the subject 140 while the subject 140 is engaging in physical activity. The real-time data 118a may include vertical position, lateral position, acceleration, orientation, etc., as well as bioelectrical information. The bioelectrical information may include muscle activity signals, heart-rate signals, etc., as described further, below. As described in more detail below, with regard to FIG. 3A-D, the digital platform 110 may receive the real-time data 118a from a performance monitor 300 worn by the subject 140 during activity.

In addition to real-time data 118a, the interactive user interface 112 may present selected athletic performance data 118b, such as a personal best metric or a record-setting metric, to compare the subject 140 with an external measure of activity. The selected performance data 118b may also include a range of values within which the subject 140 is less likely to sustain an injury while engaging in physical activity. In some embodiments, an implementation of machine learning determines the range of data values, as described in more detail, below.

In some embodiments, the data 118 includes historical performance data 118c, collected from the subject 140 over a given period of time, such as during a period of peak condition, or during a period preceding an injury. The interactive user interface 112 may display the historical performance data 118c alongside other data 118. The user 102 may select and modify data 118 as desired.

While the real-time data 118a is collected from the subject 140 directly, the data 118 may include aggregated performance data 118d collected from a number of anonymized subjects, subsequent to processing to provide useful indicators for the subject 140. For example, aggregated performance data 118d may provide correlations between various measured parameters of the real-time data 118a and likelihood of injury, such as asymmetric load on one hamstring 144, uneven exertion between two legs 142, etc.

To pair a subject 140 with data 118a-d, the subject's face 148 may be recognized by the digital platform 110 through facial recognition 160, as shown in FIG. 1B. To identify the subject 140, the digital platform 110 may capture a facial recognition image 162 showing the subject's face 148. The digital platform 110, in turn, may assign a number of landmarks 164 on the facial recognition image 162, which are subsequently used to create a unique feature map 166.

In some embodiments, the interactive user interface 112 allows a user 102 to manipulate the augmented reality environment by selecting the type of data 118 to be presented and the manner of its presentation in a way most favorable for the user 102. FIG. 1A shows data 118 being displayed as a set of rotating dials, responding in real-time to direct measurements (as in real-time data 118a) or to comparisons to other forms of data 118. For example, a rotating dial may range from 0%-100% of historical performance data 118c values, or it may display a comparison of aggregated performance data 118d as a function of time for a standardized exercise routine, as may be required for a physical therapy regimen.

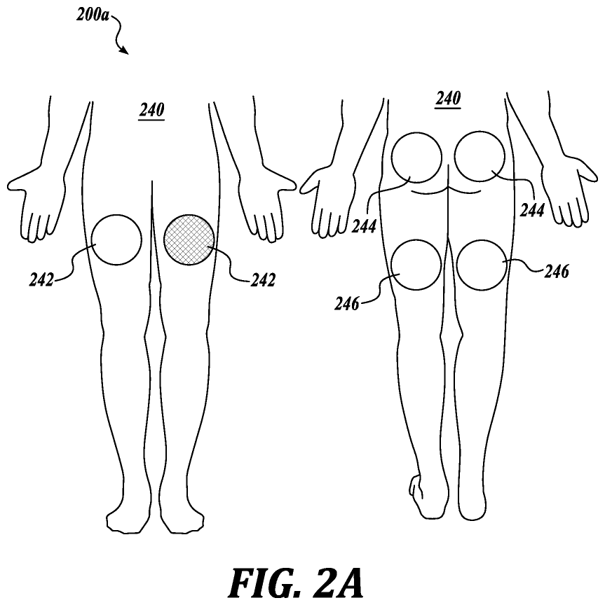

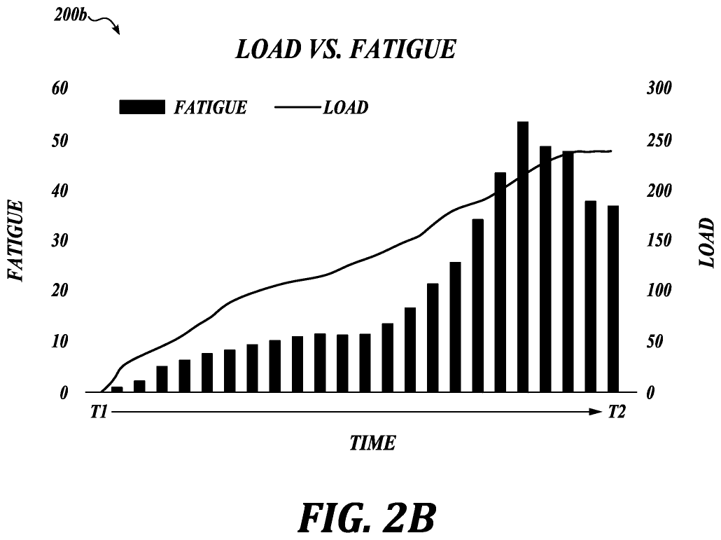

FIGS. 2A-2B show two additional visualization schemes that may be selected by the user 102 to provide an intuitive augmented reality environment through the interactive user interface 112. In some embodiments, as shown in FIG. 2A, real-time data 118a is displayed as a color-map 200a superimposed on real-time image 120 (e.g., a still image or a video) of the subject. For example, the region of the image corresponding to a measured muscle group, such as the leg 142, glute 144, or hamstring 146, may be colored either green, yellow, or red, to indicate the likelihood of fatigue-related injury. For example, if a subject 240 is favoring the left leg when performing a jumping motion, real-time data 118a measured for the leg may be represented by a red colored region 242 positioned over the image of the subject 240. If, for example, the subject 240 is not experiencing any indications of fatigue, injury, or strain on other muscle groups that information may be represented by green colored regions over each respective muscle group, such as the glutes 244 or the hamstrings 246. Other color or symbol schemes are also possible.

Additionally or alternatively to the color-map 200a shown in FIG. 2A, FIG. 2B illustrates biometric data that, in some embodiments, are presented as a time-graph 200b. The time-graph 200b may be included in the interactive user interface 112 as a way to provide the user 102 with a running view of real-time data 118a. In some embodiments, the time-graph 200b includes multiple types of real-time data 118a, including, but not limited to fatigue, load, and heart rate (in BPM, for example). The time-graph 200b may include other types of data 118, such as historical performance data 118c or aggregated performance data 118d, as a comparison against real-time data 118a to judge efficacy of the activity. As an example of a fatigue-related scenario, real-time data 118a showing a sudden increase in the heart rate that is unrelated or is weakly related to the level of muscle exertion may indicate an onset of fatigue.

FIG. 3A is a schematic diagram of the digital platform 110 in accordance with the present disclosure. In some embodiments, the digital platform 110 includes a camera 330, a logic engine 342, a communications module 341 (labeled “COM MODULE”), a display 310, and a wireless transceiver 335. As previously described, the camera 330 may provide the images 120 (e.g., video or still images) and facial recognition 160 to identify the subject 140. The communication module 341, in communication with the logic engine 342 and the performance monitor controller 305 (also referred to as the controller 305) may receive the real-time data 118a as well as other data 118 for inclusion in the interactive user interface 112, presented on the display 310. In some embodiments, the system 100 employs machine learning and/or other artificial intelligence to detect patterns and trends in the subject's heart response, muscle responses, orientation(s), acceleration(s), etc. As explained above, different combinations and rates of change of these real time data 118a, and their comparison to the selected performance data 118b, historical performance data 118c, and/or aggregated performance data 118d provides an indication of fatigue. In many embodiments, the fatigue is related to the probability of injury of the subject.

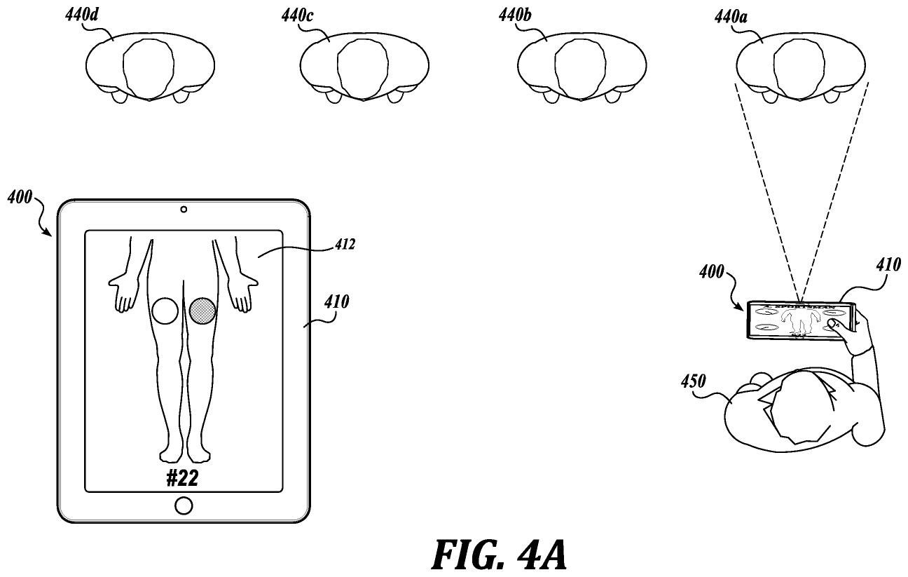

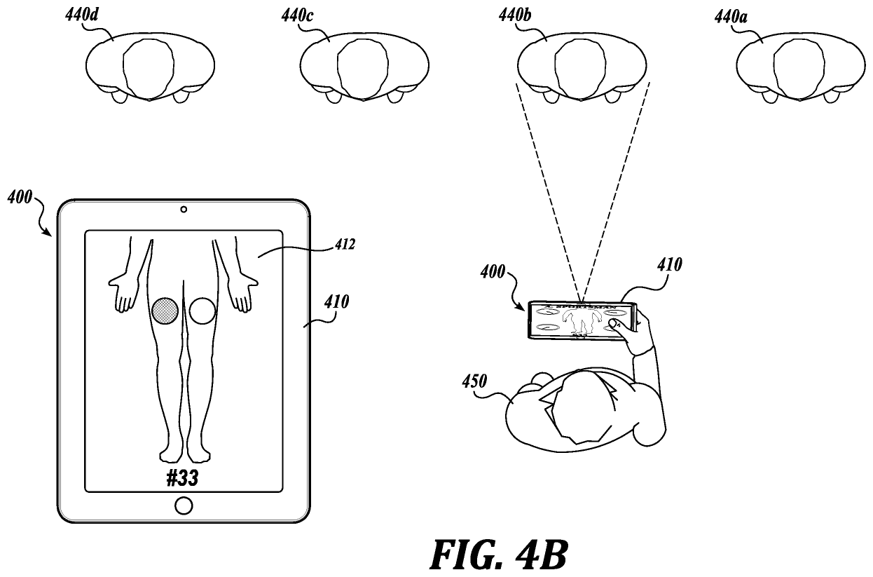

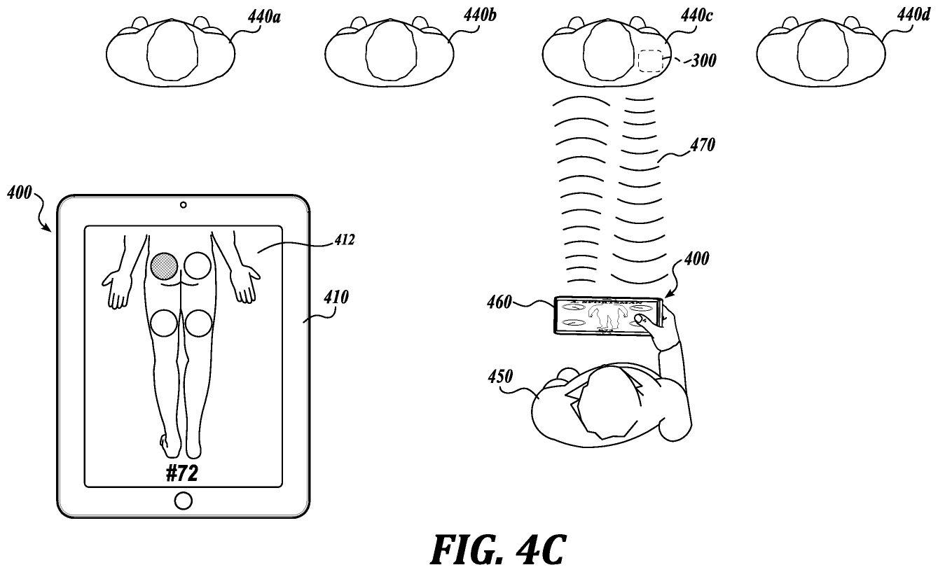

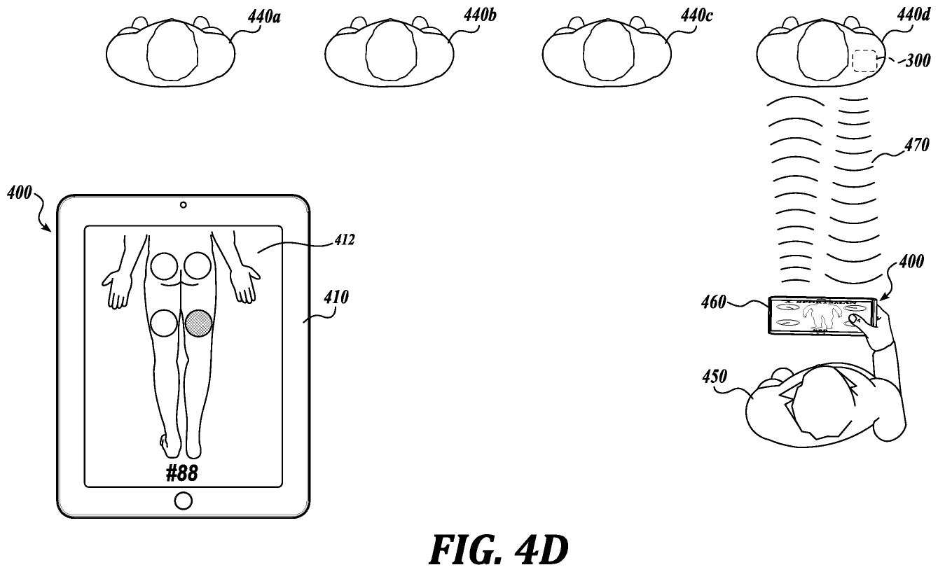

In some embodiments, the system can employ cloud learning that enables the subject 140, user 102, and others to evaluate performance and compare performance to other subjects, including anonymous subjects. The digital platform 110 may communicate with the controller 305 wirelessly, via the wireless transceiver 335, which may include Bluetooth and RFID capabilities. As discussed further with regard to FIG. 4A-D, this approach may permit identification of a subject 140 without facial recognition 160 if a subject's face 148 is not recognized or has not been added to a database.

In general, the word “engine,” as used herein, refers to logic software and algorithms embodied in hardware or software instructions, which can be written in a programming language, such as C, C++, COBOL, JAVA™, PHP, Perl, HTML, CSS, JavaScript, VBScript, ASPX, Microsoft .NET™, PYTHON, and/or the like. An engine may be compiled into executable programs or written in interpreted programming languages. Software engines may be callable from other engines or from themselves. Generally, the engine described herein refers to logical modules that can be merged with other engines, or can be divided into sub engines. The engines can be stored in any type of computer readable medium or computer storage device and be stored on and executed by one or more general purpose computers, thus creating a special purpose computer configured to provide the engine or the functionality thereof.

FIGS. 3B-3D are schematics of a performance monitoring system in accordance with the present disclosure. Referring to FIG. 3D, the controller 305 can include certain hardware and software components similar to those described above with reference to FIG. 3A. For example, the controller 305 can include a CPU 331, memory 333, and a wireless transmitter 332 (e.g., Bluetooth transmitter) over which the controller 305 communicates with the digital platform 110. Therefore, in operation, the sensors 323 may communicate their corresponding real-time data 118a (the measured data) to the digital platform 110 through the wireless communication 332 of the controller 305. In some embodiments, the controller 305 can be packaged in a water-resistant, resilient housing 342 having a small form factor.

Referring to FIGS. 3B and 3C, the controller 305 can be embedded within the subject's clothing, such as a shirt 345a and pants 345b (collectively “clothing 345”). In other embodiments, the controller 305 can be inserted into a pocket 343 in the subject's clothing and/or attached using hook-and-loop fasteners, snap, snap-fit buttons, zippers, etc. In some embodiments, the controller 305 can be removable from the clothing 345, such as for charging the controller 305. In other embodiments, the controller 305 can be permanently installed in the athlete's clothing 345.

Referring to FIGS. 3B-3D together, the controller 305 may be operably coupled to electrocardiogram (ECG) sensors 323a, electromyography (EMG) sensors 323b, an orientation sensor 323c (FIG. 3B; such as, a gyroscope), and an acceleration sensor 323d (FIG. 3B; such as, an accelerometer) that are carried at various locations on the subject's clothing 345. The sensors 323 can be connected to the controller 305 using thin, resilient flexible wires (not shown) and/or conductive thread (not shown) woven into the clothing 345. The gauge of the wire or thread can be selected to optimize signal integrity and/or reduce electrical impedance.

The ECG and EMG sensors 323a and 323b may include dry-surface electrodes distributed throughout the subject's clothing 345 and positioned to make necessary skin contact beneath the clothing along predetermined locations of the body. In some embodiments, the sensors can include an optical detector, such as an optical sensor for measuring heart rate. The fit of the clothing can be selected to be sufficiently tight to provide continuous skin contact with the individual sensors 323a and 323b, allowing for accurate readings, while still maintaining a high-level of comfort, comparable to that of traditional compression fit shirts, pants, and similar clothing. In various embodiments, the clothing 345 can be made from compressive fit materials, such as polyester and other materials (ex. Elastaine) for increased comfort and functionality. In some embodiments, the controller 305 and the sensors 323 can have sufficient durability and water-resistance so that they can be washed with the clothing 345 in a washing machine without causing damage. In these and other embodiments, the presence of the controller 305 and/or the sensors 323 within the clothing 345 may be virtually unnoticeable to the subject. In one aspect of the technology, the sensors 323 can be positioned on the subject's body without the use of tight and awkward fitting sensor bands. In general, traditional sensor bands are typically uncomfortable for a subject, and subjects can be reluctant to wear them.

The ECG sensors 323a can include right arm RA, left arm LA, and right leg RL (floating ground) sensors positioned on the subject's chest and waist. The EMG sensors 323b can be positioned adjacent to targeted muscle groups, such as the large muscle groups of the pectoralis major, rectus abdominis, quadriceps femoris, biceps, triceps, deltoids, gastrocnemius, hamstring, and latissimus dorsi. The EMG sensors 323b can also be coupled to floating ground near the subject's waist or hip.

The orientation and accelerations sensors 323c and 323d can be disposed at a central position 349 located between the athlete's shoulders and upper back region. In some embodiments, the central, upper back region can be an optimal location for placement of the orientation and acceleration sensors 323c and 323d because of the relatively small amount of muscle tissue in this region of the body, which prevents muscle movement from interfering with the accuracy of the orientation and acceleration readings. In other embodiments, the orientation sensor 323c and/or the acceleration sensor 323d can be positioned centrally on the user's chest, tail-bone, or other suitable locations of the body. In various embodiments, the orientation and acceleration sensors 323c and 323d can be positioned adjacent the controller 305, or integrated into the same packaging (e.g., housing) 322 as controller 305, as shown in FIG. 3B. In other embodiments, the orientation sensor 323c and/or the acceleration sensor 323d can be positioned at other locations. In use, the acceleration and orientation sensors 323a and 323b can detect 3D orientation and 3D acceleration of the central position 349 (corresponding, e.g., to a center of mass).

In one aspect of this embodiment, the use of a single orientation sensor and a single acceleration sensor can reduce computational complexity of the various analytics 110 (FIG. 1B) produced by the system 100 (FIG. 1A). In particular, a reduced set of orientation and acceleration data may be sufficient for detecting various indicators of fatigue and other performance characteristics in conjunction with the other real-time data 118a (FIG. 1A) collected from the other sensors 323 and based on other analytics derived in previous live sessions, as described previously. In other embodiments, however, the performance monitor 300 can include multiple acceleration sensors and/or orientation sensors, such as for detecting acceleration and/or orientation of one or more of the subject's limbs.

Referring back to FIG. 3B, the controller 305 and the sensors 323 can be powered by a power device 348, such as a rechargeable battery carried within the controller's housing 322. In some embodiments, the power device 348 can be a kinetic energy device (having, e.g., piezoelectrics) configured to convert and store energy generated by the subject 140 (FIG. 1A) while wearing the clothing 345 and/or while the clothing 345 is being cleaned in a washing machine and/or a dryer.

In some embodiments, the performance monitor 300 (FIG. 1A) does not include the pants 345b and/or includes sensors positioned in other garments in addition to or in lieu of the pants 345b, such as shorts, a headband, socks, shoes, etc. In some embodiments, the performance monitor 300 can include other input and/or output components 344, such as a feedback device (e.g., a speaker or a vibration generator) that provides real-time feedback to the athlete while wearing the clothing. For example, the feedback can include a series of audible beeps and/or vibrations that increase in frequency as the athlete is approaching a state of fatigue. In these and other embodiments, the controller 305 can be configured to directly communicate with a Bluetooth headset for voice communication with the user 102, to download real-time data stored in the memory 333 after completion of a live session (e.g., for further analysis), and/or to perform other functions. In some embodiments, the performance monitor 300 can include a magnetometer for self-calibration of the orientation sensor 323c and/or the accelerometer 323d. A magnetometer may also be used in conjunction with or in lieu of the orientation sensor for providing orientation data.

In additional or alternate embodiments, the performance monitor 300 (FIGS. 3B-C) can include a separate controller 346 worn on the subject's pants 345b. The separate controller 346 can be similar to the controller 305 worn on the subject's shirt 345a and is connected to the individual sensors 323 located on the pants 345b. The separate controller 346 can be configured to communicate with the controller 305 and/or with the digital platform 110 (FIG. 3A) independent of the controller 305.

FIGS. 4A-D are schematic diagrams of the digital platform 410 in accordance with the present disclosure. In some embodiments, the system 400 may be repositioned to provide analytics of multiple subjects 440a-d engaging in similar or different activities while individually wearing performance monitors. A user 450 (such as a coach, trainer, therapist, doctor, etc.) may use a digital platform 410 as previously described to identify a subject 440a through facial recognition, as shown in FIG. 4A, and observe the subject 440a through an augmented reality environment presented in an interactive user interface 412. As previously described, the interactive user interface 412 may present an image of the subject 440a along with a color-map of real-time data 118a superimposed thereon. In some embodiments, the image of the subject includes videos that depend-on or depict patterns and trends determined by the artificial intelligence. These images may be replayed by the user 450. In some embodiments, as shown in FIG. 4B, the user 450 may reposition the digital platform 410 from the subject 440a to a second subject 440b. The digital platform may receive a command to disassociate from the earlier subject 440a and to identify and analyze the second subject 440b. Alternatively, the digital platform 410 may automatically identify all available subjects 440a-d and provide the user 450 with an option to select an augmented reality interface for one or more subjects 440 manually. In some embodiments, the digital platform 410 may prompt the user 450 when a new subject 440 is detected. In some embodiments, the interactive user interface 412 provides an augmented reality environment to aid the user 450 in guiding the second subject 440b during the activity.

As shown in FIG. 4C, in some embodiments, the user 450 may be unable to identify a subject 440c using facial recognition and will instead direct the digital platform 410 to communicate wirelessly with the performance monitor 300 worn by the subject 440c. The digital platform 410 may communicate wirelessly 470 via a wireless transceiver 460, as previously described, for example, using Bluetooth or RFID technology. A subject's activity may be observed from multiple angles both in front of and behind the subject 440c. If the user 450 wishes to observe the subject from behind, for example, the digital platform 410 may recognize that the user 450 is standing behind the subject 440c, and display data from sensors measuring muscle groups located on the backside of the subject 440c. In a similar fashion, the digital platform 410 may populate the interactive user interface 412 with selected performance data 118b, historical performance data 118c, and/or aggregated performance data 118d corresponding to the muscle-groups visible at the angle from which the user 450 is observing the subject 440c. In some embodiments, a similar approach to reacquiring a subject of observation as in FIG. 4B is implemented, only using wireless communication with the performance monitor 300 worn by the subject 440d, as shown in FIG. 4D.

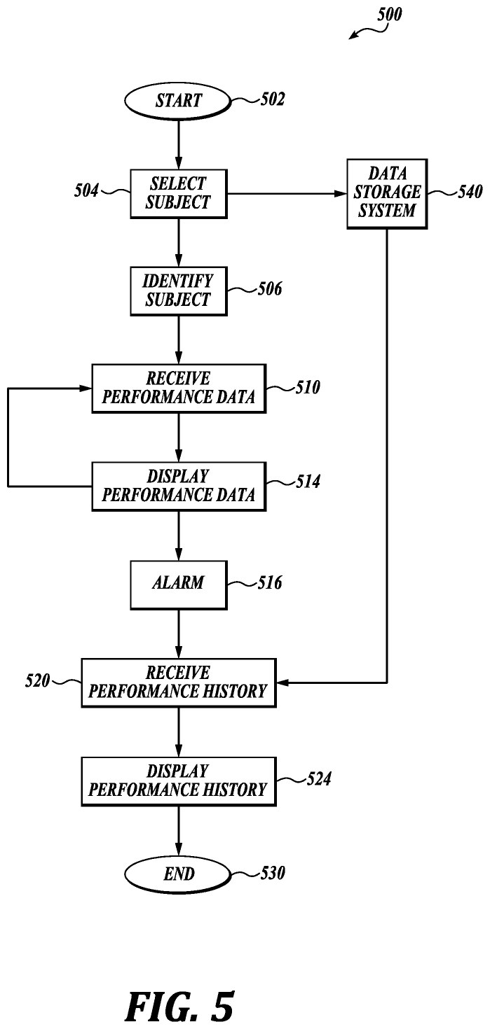

FIG. 5 is a flowchart of a method 500 of assessing athletic performance in real-time through an augmented reality environment using the system 100. In some embodiments, the method may include additional steps or may be practiced without all steps illustrated in the flow chart. In some embodiments, the method 500 starts in block 502 and proceeds to block 504, where a subject 140 is selected by the user 102. In block 506, the digital platform 110 identifies the subject 140. As previously described, the digital platform 110 may identify the subject 140 using facial recognition or through wireless communication including, but not limited to Bluetooth and RFID pairing with the performance monitor 300 being worn by the subject 140. Following identification, the method 500 proceeds to block 510, wherein the digital platform receives real-time data 118a from the performance monitor 300 and processes it for presentation in an augmented reality environment, shown in block 514. The augmented reality environment may be a part of the interactive user interface 112, which may be updated in real-time for the duration of the activity as shown by the loop linking block 514 with block 510.

In some embodiments, the method 500 includes the digital platform 110 referring to a data storage system 540 that receives the information gathered in block 504. The data storage system 540, as previously described, can provide aggregated performance data 118d from multiple anonymous subjects, historical performance data 118c from the subject 140, as well as analytics and model-predictive adjustments of indicators of fatigue. In such cases, the method 500 displays data 118 in the augmented reality environment as part of the interactive user interface 112.

In some embodiments, the interactive user interface 112 presents visual or auditory feedback to the user 450 when real-time data 118a indicates a high likelihood of an adverse outcome, as shown in block 516. For example, the performance monitor 300 may detect a bioelectric signal indicating a high likelihood of hamstring injury, based on model predictions, and the interactive user interface 112 may provide a blinking indicator over the relevant muscle group on the subject 440. For example, the digital platform 410 may provide feedback when the performance monitor 300 detects that a left hamstring is bearing an excess load, based on models of healthy and effective load balancing. In some embodiments, the user 450 designates values of real-time data 118a for which feedback will be provided. In some embodiments, the values for which feedback will be provided are designated automatically via a model-prediction, as previously described.

Many embodiments of the technology described above may take the form of computer- or controller-executable instructions, including routines executed by a programmable computer or controller. Those skilled in the relevant art will appreciate that the technology can be practiced on computer/controller systems other than those shown and described above. The technology can be embodied in a special-purpose computer, application specific integrated circuit (ASIC), controller or data processor that is specifically programmed, configured or constructed to perform one or more of the computer-executable instructions described above. Of course, any logic or algorithm described herein can be implemented in software or hardware, or a combination of software and hardware.

From the foregoing, it will be appreciated that specific embodiments of the technology have been described herein for purposes of illustration, but that various modifications may be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

I/We claim:

1. An augmented reality system for real-time assessment of an athletic performance, the system comprising:
a digital platform including a display, at least one camera, and a communications module;
a performance monitor carried by a garment and configured to communicate wirelessly with the digital platform;
a logic engine; and
an interactive user interface, presenting real-time data and images of the athletic performance in an augmented reality environment, the real-time data and images including—
images obtained by the at least one camera; and
athletic performance data received from the performance monitor.

2. The system of claim 1, wherein the interactive user interface further presents:
historical performance data; and
aggregated performance data.

3. The system of claim 2, wherein historical performance data comprises real-time data collected from an identified individual over a period of time.

4. The system of claim 2, wherein aggregated performance data comprises real-time data collected from a plurality of anonymized individuals.

5. The system of claim 4, further comprising at least one predictive model indicating a likelihood of one or more of fatigue or injury in correlation to real-time data.

6. The system of claim 1, wherein the performance monitor comprises:
a plurality of sensors positioned in one or more regions of the garment configured to measure signals generated during the athletic performance; and
a performance monitor controller, comprising:
an onboard analytics module configured to receive and process signals from the plurality of sensors; and
an onboard communications module in wireless communication with the digital platform.

7. The system of claim 6, wherein the performance monitor comprises sensors to measure orientation, acceleration, heart response, and muscle response.

8. The system of claim 1, wherein the logic engine comprises an implementation of machine learning.

9. The system of claim 1, wherein the augmented reality system further comprises a data storage system in communication with the digital platform and the performance monitor, the data storage system including an implementation of machine learning.

10. A method of assessing athletic performance in real-time through an augmented reality environment, the method comprising:
selecting a subject of observation;
identifying the subject of observation using a digital platform;
presenting an augmented reality environment including an interactive user interface and data including—
images of the subject of observation collected via a camera; and
real-time data collected via a performance monitor; and
receiving commands from a user via the interactive user interface, wherein the commands modify one or more of the interactive user interfaces, the operation of the performance monitor, the selection of the subject of observation, and the presentation of data.

11. The method of claim 10, wherein the data further comprise:
historical performance data collected from the subject of observation; and
aggregated performance data collected from multiple anonymized subjects.

12. The method of claim 10, further comprising:
accessing real-time analytics provided by an external data storage system; and
processing the real-time data using model-predictions of athletic performance.

13. The method of claim 10, further comprising:
identifying multiple subjects engaging in simultaneous athletic performances;
presenting one or more available subjects via the interactive user interface;
prompting a selection of one or more of the available subjects for observation in real-time.

14. The method of claim 10, further comprising:
indicating, via a visual or auditory signal, when the subject of observation has a high likelihood of adverse outcome from athletic performance.

15. An augmented reality system for real-time assessment of a physical rehabilitation treatment, the system comprising:
a digital platform including a display, at least one camera, and a communications module;
a performance monitor carried by a garment and configured to communicate wirelessly with the digital platform;
a logic engine; and
an interactive user interface, presenting real-time data and images of the physical rehabilitation treatment in an augmented reality environment, the real-time data and images including—
images obtained by the at least one camera; and
physical rehabilitation treatment data received from the performance monitor.

16. The system of claim 15, wherein the interactive user interface further presents:
historical performance data; and
aggregated performance data.

17. The system of claim 16, wherein historical performance data comprises real-time data collected from an identified individual over a period of time.

18. The system of claim 16, wherein aggregated performance data comprises real-time data collected from a plurality of anonymized individuals.

19. The system of claim 16, further comprising at least one predictive model indicating a likelihood of one or more of fatigue or injury in correlation to real-time data.

20. The system of claim 15, wherein the performance monitor comprises:
a plurality of sensors positioned in one or more regions of the garment configured to measure signals generated during the athletic performance; and
a performance monitor controller, comprising:
an onboard analytics module configured to receive and process signals from the plurality of sensors; and
an onboard communications module in wireless communication with the digital platform.

21. The system of claim 20, wherein the performance monitor comprises sensors to measure orientation, acceleration, heart response, and muscle response.

22. The system of claim 15, wherein the logic engine comprises an implementation of machine learning.

23. The system of claim 15, wherein the augmented reality system further comprises a data storage system in communication with the digital platform and the performance monitor, the data storage system including an implementation of machine learning.
